(19) United States
(12) Patent Application Publication
(10) Pub. No.: US 2015/0259403 A1
(43) Pub. Date: Sep. 17, 2015

(54) Methods and Pharmaceutical Compositions for Treatment of Gastrointestinal Stromal Tumors

(71) Applicants: INSERM (INSTITUT NATIONAL DE LA SANTÉ ET DE LA RECHERCHE MÉDICALE), Paris (FR); UNIVERSITÉ DE MONTPELLIER I, Montpellier Cedex 2 (FR); UNIVERSITÉ MONTPELLIER 2 SCIENCES ET TECHNIQUES, Montpellier (FR)

(72) Inventors: Pascal De Santa Barbara, Montpellier Cedex (FR); Jean-Francois Guichou, Montpellier (FR)

(21) Appl. No.: 14/433,689

(22) PCT Filed: Oct. 10, 2013
(86) PCT No.: PCT/EP2013/071182

§ 371 Date: Apr. 6, 2015

Related U.S. Application Data
(60) Provisional application No. 61/772,985, filed on Mar. 5, 2013.

(30) Foreign Application Priority Data
Oct. 10, 2012 (EP) 12306238.2

(51) Int. Cl. C07K 16/18; G01N 33/574; C12Q 1/68; G01N 33/50; A61K 38/02; A61K 31/713

(52) U.S. Cl. CPC C07K 16/18; A61K 38/02; A61K 31/713; C12Q 1/6886; G01N 33/5011; G01N 33/57446; C07K 2317/76; C12Q 2600/158; C12Q 2600/16; G01N 2333/47

(57) ABSTRACT

The present invention relates to methods and compositions for the treatment of gastrointestinal stromal tumors.

### FIELD OF THE INVENTION

The present invention relates to methods and compositions for the treatment of gastrointestinal stromal tumors.

### BACKGROUND OF THE INVENTION

Gastrointestinal stromal tumors (GISTs) are the most common mesenchymal neoplasms of the gastrointestinal tract (Corless et al., 2011) and are highly resistant to conventional chemotherapy and radiotherapy. These tumors are characterized by the presence of activating mutations in KIT (75-80% frequency) or Platelet-Derived Growth Factor Receptor Alpha (PDGFRA) (5-10% of tumors), two genes encoding receptors for growth factors that are normally activated only in specific situations (Hirato et al., 1998). Imatinib mesylate, a small-molecular tyrosine kinase inhibitor that targets phosphorylation/activation of KIT and PDGFRA and also constitutively activated KIT and PDGFRA proteins, has proven efficient in GIST treatment (Joensuu et al., 2001; Tuveson et al., 2001); however, resistance to such therapy is increasing. Therefore, the development of new-targeted therapies is strongly encouraged (Renouf et al., 2009).

It is now largely documented that post-regulatory RNA events play crucial roles in modulating differentiation and remodeling of smooth muscle tissues (Xin et al., 2009; Notarnicola et al., 2012). RNA-protein complexes control multiple steps of this process, including mRNA cellular localization, splicing, translational regulation and degradation (St Johnston, 2005). For instance, the expression of an alternative splicing isoform of the natural killer (NK) cell receptor NKp30 correlates with the prognosis of GISTs, independently from the KIT mutations (Delahaye et al., 2011).

Moreover, RNA-binding proteins (RBPs), which play important roles in regulating RNA metabolism, may also be deregulated in diseases, particularly in cancers during the initiation and progression phases (van Kouwenhove et al., 2011). The RNA Recognition Motif (RRM) proteins form a large RBP family which includes also RNA-Binding Protein for Multiple Splicing-2 (RBPMS2), an early marker of gastrointestinal smooth muscle precursor cells that the inventors identified recently (Notamicola et al., 2012). The inventors showed that ectopic expression of RBPMS2 in differentiated SMCs hinders their ability to contract, favors their proliferation and leads to their dedifferentiation, demonstrating that RBPMS2 expression must be tightly regulated to avoid SMC dedifferentiation (Notarnicola et al., 2012).

These results and the fact that GISTs are thought to arise from Interstitial Cells of Cajal (ICCs) or from a mesenchymal precursor that is common to ICCs and smooth muscle cells (SMCs) (Sanders et al., 2006; Le Guen et al., 2009; Faure and de Santa Barbara, 2011) prompted the inventors to examine the mRNA and protein expression of RBPMS2 in different categories of human adult GISTs and in the GIST882 cell line.

### SUMMARY OF THE INVENTION

The present invention relates to a compound which is selected from the group consisting of RBPMS2 dimerization inhibitors, RBPMS2 antagonists or RBPMS2 expression inhibitors for use in the treatment of gastrointestinal stromal tumors (GISTs) in a subject in need thereof.

### DETAILED DESCRIPTION OF THE INVENTION

The inventors analyzed the mRNA and protein expression of RNA-Binding Protein with Multiple Splicing-2 (RBPMS2), an early marker of gastrointestinal SMC precursors, in human GISTs (n=23) by in situ hybridization, quantitative RT-PCR analysis and immunohistochemistry. The inventors surprisingly demonstrated that the RBPMS2 mRNA level in GISTs was 42-fold higher than in control gastrointestinal samples (p<0.001). The inventors also demonstrated that RBPMS2 expression was not correlated with KIT and PDGFRA expression levels, but was higher in GISTs harboring KIT mutations than in tumors with wild type KIT and PDGFRA or in GISTs with PDGFRA mutations that were characterized by the lowest RBPMS2 levels. Moreover, RBPMS2 levels were 64-fold higher in GIST samples with high risk of aggressive behavior than in adult control gastrointestinal samples and 6.2-fold higher in high risk than in low risk GIST specimens. RBPMS2 protein level was high in 87% of the studied GISTs independently of their histological classification. By inhibiting the KIT signaling pathway in GIST882 cells, the inventors show that RBPMS2 expression is independent of KIT activation. RBPMS2 is then up-regulated in GISTs compared to normal adult gastrointestinal tissues, indicating that RBPMS2 represent a new diagnostic marker for GISTs and a target for cancer therapy.

### Therapeutic Methods and Uses

Accordingly, the present invention relates to a compound which is selected from the group consisting of RBPMS2 dimerization inhibitors, RBPMS2 antagonists or RBPMS2 expression inhibitors for use in the treatment of gastrointestinal stromal tumors (GISTs) in a subject in need thereof.

As used herein, the term “subject” denotes a mammal. In a preferred embodiment of the invention, a subject according to the invention refers to any subject (preferably human) afflicted with gastrointestinal stromal tumors (GISTs).

The method of the invention may be performed for any type of gastrointestinal stromal tumors (GISTs) such as revised in the World Health Organisation Classification of gastrointestinal stromal tumors (GISTs) and selected from the group: 8936/1 (http://www.pubcan.org/searchresults.php?action=search&icdo=8936/1&id=600). Gastrointestinal stromal tumors (GISTs) as used herein refers to tumors that occurs in the gastrointestinal (GI or digestive) tract, including the esophagus, stomach, gallbladder, liver, small intestine, colon, ampulla vater, rectum, omentum, anus, and lining of the gut.

The term “low risk GISTs” or “low risk tumor” has its general meaning in the art and refers to GISTs or tumor with low risk of clinically aggressive behavior. The term “low risk GISTs” refers to GISTs with low level of progression.

The term “high risk GISTs” or “high risk tumor” has its general meaning in the art and refers to GISTs or tumor with high risk of clinically aggressive behavior. The term “high risk GISTs” refers to GISTs with high level of progression.

As used herein, the term “RBPMS2” has its general meaning in the art and refers to RNA-Binding Protein for Multiple Splicing-2 (SEQ ID NO:1) (Notarnicola et al., 2012). The term “RBPMS2” refers to the RNA-Binding Protein RBPMS2, an early marker of SMC precursor cells and that ectopic expression of RBPMS2 in differentiated SMCs conducts to their dedifferentiation and triggers their proliferation.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., RBPMS2) modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene.

In one embodiment, the compound of the invention is a RBPMS2 dimerization inhibitor.

As used herein, the term “RBPMS2 dimerization inhibitor” refers to a compound that selectively prevents or blocks RBPMS2 dimerization. The term “RBPMS2 dimerization inhibitor” refers to a compound that targets the residue Leucine 49 of RBPMS2 protein and blocks RBPMS2 dimerization. The term “RBPMS2 dimerization inhibitor” also refers to a compound that targets the RRM-homodimeriztion motif (amino acid residues 47-50 of the SEQ ID NO:1). Typically, a RBPMS2 dimerization inhibitor is a small organic molecule, a peptide, a polypeptide, an aptamer or an intra-antibody.

In one embodiment, the compound of the invention is a RBPMS2 antagonist.

The term “RBPMS2 antagonist” refers to a compound that selectively blocks or inactivates the RBPMS2. The term “RBPMS2 antagonist” also refers to a compound that selectively blocks the binding of RBPMS2 to RNAs via its RRM domain. The term “RBPMS2 antagonist” also refers to a compound that selectively blocks RBPMS2 binding to Noggin, inducing Noggin up-regulation and then inhibiting BMP signalling. As used herein, the term “selectively blocks or inactivates” refers to a compound that preferentially binds to and blocks or inactivates RBPMS2 with a greater affinity and potency, respectively, than its interaction with the other sub-types or isoforms of the RBPMS family. Compounds that prefer RBPMS2, but that may also block or inactivate other nuclear receptor sub-types, as partial or full antagonists, are contemplated. Typically, a RBPMS2 antagonist is a small organic molecule, a peptide, a polypeptide, an aptamer or an intra-antibody.

In another embodiment, the RBPMS2 dimerization inhibitor or RBPMS2 antagonist of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against RBPMS2 of the invention as above described, the skilled man in the art can easily select those inhibiting RBPMS2 dimerization or inhibiting RBPMS2.

In one embodiment, the compound of the invention is an inhibitor of RBPMS2 expression.

Inhibitors of RBPMS2 expression for use in the present invention may be based on antisense oligonucleotide constructs. Anti-sense oligonucleotides, including anti-sense RNA molecules and anti-sense DNA molecules, would act to directly block the translation of RBPMS2 mRNA by binding thereto and thus preventing protein translation or increasing mRNA degradation, thus decreasing the level of RBPMS2 proteins, and thus activity, in a cell. For example, antisense oligonucleotides of at least about 15 bases and complementary to unique regions of the mRNA transcript sequence encoding RBPMS2 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of RBPMS2 expression for use in the present invention. RBPMS2 gene expression can be reduced by contacting the subject or cell with a small double stranded RNA (d5RNA), or a vector or construct causing the production of a small double stranded RNA, such that RBPMS2 expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of RBPMS2 expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of RBPMS2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of RBPMS2 expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing RBPMS2. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Chiffon, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In one embodiment, the present invention relates to a compound which is selected from the group consisting of RBPMS2 dimerization inhibitors, RBPMS2 antagonists or RBPMS2 expression inhibitors for use in the prevention or treatment of high risk gastrointestinal stromal tumors (GISTs) in a subject in need thereof.

In one embodiment, the present invention relates to a method of treating gastrointestinal stromal tumors (GISTs) in a subject in need thereof, comprising the step of administering to said subject a compound which is selected from the group consisting of RBPMS2 dimerization inhibitors, RBPMS2 antagonists or RBPMS2 expression inhibitors.

In one embodiment, the present invention relates to a method of preventing or treating high risk gastrointestinal stromal tumors (GISTs) in a subject in need thereof, comprising the step of administering to said subject a compound which is selected from the group consisting of RBPMS2 dimerization inhibitors, RBPMS2 antagonists or RBPMS2 expression inhibitors.

### Pharmaceutical Composition

The compound of the invention may be used or prepared in a pharmaceutical composition.

In one embodiment, the invention relates to a pharmaceutical composition comprising the compound of the invention and a pharmaceutical acceptable carrier for use in the treatment of gastrointestinal stromal tumors (GISTs) in a subject in need thereof.

Typically, the compound of the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In one embodiment, the present invention relates to a pharmaceutical composition comprising the compound of the invention and a pharmaceutical acceptable carrier for use in the prevention or treatment of high risk gastrointestinal stromal tumors (GISTs) in a subject in need thereof.

In another embodiment, the present invention relates to a pharmaceutical composition comprising the compound of the invention and a pharmaceutical acceptable carrier for use in prevention of progression of low risk gastrointestinal stromal tumors (GISTs) to high risk gastrointestinal stromal tumors (GISTs) in a subject in need thereof.

### Screening Method

In a further aspect, the present invention relates to a method of screening a candidate compound for use as a drug for the treatment of gastrointestinal stromal tumors (GISTs) in a subject in need thereof, wherein the method comprises the steps of: i) providing candidate compounds and ii) selecting candidate compounds that blocks the action of RBPMS2.

In a further aspect, the present invention relates to a method of screening a candidate compound for use as a drug for the treatment of gastrointestinal stromal tumors (GISTs) in a subject in need thereof, wherein the method comprises the steps of:
(i) providing a RBPMS2, providing a cell, tissue sample or organism expressing the RBPMS2,
(ii) providing a candidate compound such as small organic molecule, intra-antibodies, peptide or polypeptide,
(iii) measuring the activity of the RBPMS2,
(iv) and selecting positively candidate compounds that blocks RBPMS2 dimerization, blocks the action of RBPMS2 or inhibits RBPMS2 expression.

Methods for measuring the activity of the RBPMS2 are well known in the art. For example, measuring the RBPMS2 activity involves measuring RBPMS2 dimerization level on the RBPMS2 cloned and transfected in a stable manner into a CHO cell line, human embryonic kidney (HEK) cell line or human GIST cell line, measuring Noggin expression level, measuring the expression level of contractile proteins, determining the hypertrophic phenotype or measuring SMC contractility level in the presence or absence of the candidate compound.

Tests and assays for screening and determining whether a candidate compound is a RBPMS2 dimerization inhibitor, RBPMS2 antagonist or RBPMS2 expression inhibitor are well known in the art. In vitro and in vivo assays may be used to assess the potency and selectivity of the candidate compounds to reduce RBPMS2 activity.

Activities of the candidate compounds, their ability to bind RBPMS2 and their ability to inhibit RBPMS2 activity may be tested using isolated smooth muscle cells (SMC) expressing RBPMS2, CHO cell line, human embryonic kidney cell line (HEK) or human GIST cell line cloned and transfected in a stable manner by the human RBPMS2.

Cells and smooth muscle cells expressing another RNA-binding protein than RBPMS2 may be used to assess selectivity of the candidate compounds.

In one embodiment, the present invention relates to a method of screening a candidate compound for use as a drug for the treatment of gastrointestinal stromal tumors (GISTs) in a subject in need thereof, wherein the method comprises the steps of:
i) providing a polypeptide comprising amino acid residues 47-50 of the SEQ ID NO:1,
ii) providing a candidate compound such as small organic molecule, intra-antibodies, peptide or polypeptide,
iv) measuring the binding of the candidate compound to the polypeptide of step i) using appropriate biophysical techniques,
v) and positively selecting candidate compounds that bind to the polypeptide of step i).

Typically, the candidate compound bind to the amino acid residues 47-50 of the SEQ ID NO:1 of the polypeptide and blocks polypeptides dimerization.

Methods for measuring the binding of the candidate agent to the polypeptide comprising amino acid residues 47-50 of the SEQ ID NO:1 are well known in the art. For example, measuring the binding of the candidate agent to said polypeptide may be performed by biophysical techniques such as binding tests and crystallography.

### Diagnostic and Prognostic Methods

A further aspect of the invention relates to a method of identifying a subject having a gastrointestinal stromal tumors (GISTs) which comprises the step of analyzing a biological sample from said subject for:

(i) determining the RBPMS2 expression level,

(ii) comparing the RBPMS2 expression level in the sample with a reference value,

(iii) detecting differential in the RBPMS2 expression level between the sample and the reference value is indicative of a subject having a gastrointestinal stromal tumors (GISTs).

Analyzing the RBPMS2 expression level may be assessed by any of a wide variety of well-known methods for detecting expression of a transcribed nucleic acid or translated protein.

In a preferred embodiment, RBPMS2 expression level is assessed by analyzing the expression of mRNA transcript or mRNA precursors, such as nascent RNA, of RBPMS2 gene. Said analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFFYMETRIX).

Advantageously, the analysis of the expression level of mRNA transcribed from the gene encoding for RBPMS2 involves the process of nucleic acid amplification, e.g., by RT-PCR (the experimental embodiment set forth in U.S. Pat. No. 4,683,202), ligase chain reaction (Barany, 1991), self sustained sequence replication (Guatelli et al., 1990), transcriptional amplification system (Kwok et al., 1989), Q-Beta Replicase (Lizardi et al., 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the RBPMS2 expression level is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for RBPMS2.

Said analysis can be assessed by a variety of techniques well known from one of skill in the art including, but not limited to, enzyme immunoassay (EIA), radioimmunoassay (RIA), Western blot analysis and enzyme linked immunoabsorbant assay (RIA).

A reference value can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the RBPMS2 expression levels (obtained according to the method of the invention) with a defined threshold value. In one embodiment of the present invention, the threshold value is derived from the RBPMS2 expression level (or ratio, or score) determined in a biological sample derived from one or more subjects having or at risk of having or developing a gastrointestinal stromal tumors (GISTs). In one embodiment of the present invention, the threshold value may also be derived from RBPMS2 expression level (or ratio, or score) determined in a biological sample derived from one or more subjects having or at risk of having or developing a gastrointestinal stromal tumors (GISTs). Furthermore, retrospective measurement of the RBPMS2 expression levels (or ratio, or scores) in properly banked historical subject samples may be used in establishing these threshold values.

In one embodiment of the invention, the reference value may consist in expression level measured in a biological sample associated with a healthy subject not afflicted with gastrointestinal stromal tumors (GISTs) or in a biological sample associated with a subject afflicted with gastrointestinal stromal tumors (GISTs).

According to the invention, a lower RBPMS2 expression level in the sample than the reference value is indicative of subject not having a gastrointestinal stromal tumors (GISTs) and a higher RBPMS2 expression level in the sample than the reference value is indicative of subject having a gastrointestinal stromal tumors (GISTs).

In another embodiment, the present invention relates to a method of determining whether the gastrointestinal stromal tumor (GIST) of a subject is a low risk tumor or a high risk tumor which comprises the step of analyzing a biological sample from said subject for:

(i) determining the RBPMS2 expression level,

(ii) comparing the RBPMS2 expression level in the sample with a reference value,

(iii) detecting differential in the RBPMS2 expression level between the sample and the reference value is indicative that the gastrointestinal stromal tumor (GIST) is a low risk tumor or a high risk tumor.

According to the invention, a lower RBPMS2 expression level in the biological sample than the reference value is indicative that the gastrointestinal stromal tumor (GIST) is a low risk tumor and higher RBPMS2 expression level in the biological sample than the reference value is indicative that the gastrointestinal stromal tumor (GIST) is a high risk tumor.

In one embodiment, the present invention relates to a compound which is selected from the group consisting of RBPMS2 dimerization inhibitors, RBPMS2 antagonists or RBPMS2 expression inhibitors for use in the prevention of progression of low risk gastrointestinal stromal tumors (GISTs) to high risk gastrointestinal stromal tumors (GISTs) in a subject in need thereof wherein the subject was being classified as having a high risk tumor by the method as above described.

A further aspect of the invention relates to a method of monitoring gastrointestinal stromal tumors (GISTs) progression by performing the method of the invention.

In one embodiment, the present invention relates to a method of treating gastrointestinal stromal tumors (GISTs) in a subject in need thereof comprising the steps of:

i) identifying a subject having a gastrointestinal stromal tumors (GISTs) by performing the method according to the invention, and

ii) administering to said subject a compound which is selected from the group consisting of RBPMS2 dimerization inhibitors, RBPMS2 antagonists or RBPMS2 expression inhibitors.

In one embodiment, the present invention relates to a method of preventing the progression of low risk gastrointestinal stromal tumors (GISTs) to high risk gastrointestinal stromal tumors (GISTs) in a subject in need thereof comprising the steps of:

i) determining whether the gastrointestinal stromal tumor (GIST) of a subject is a low risk tumor or a high risk tumor by performing the method according to the invention, and

ii) administering a compound which is selected from the group consisting of

RBPMS2 dimerization inhibitors, RBPMS2 antagonists or RBPMS2 expression inhibitors if said subject was being classified as having a high risk tumor.

The invention will be further illustrated by the following figures and examples. However, these examples and figures should not be interpreted in any way as limiting the scope of the present invention.

### FIGURES

FIG. 1: Clinicopathological features and RBPMS2 mRNA expression in the adult GIST cohort.

(A) Comparison of the RBPMS2 transcript levels in the GIST and non-tumoral samples described in A with the Mann-Whitney test (p<0.001). (B) Mean expression level of RBPMS2, KIT and PDGFRA in GISTs that were classified based on their KIT and PDGFRA mutational status. (C) Mean expression level of RBPMS2, KIT and PDGFRA in GISTs that were classified according to the risk of aggressive behavior.

FIG. 2: RBPMS2 mRNA expression and regulation in the GIST882 cell line.

(A) Analysis of RBPMS2 expression by quantitative RT-PCR in GIST882, HeLa, LNCaP, 1321N1 and PC3 cells. RBPMS2 is strongly expressed in GIST882 and HeLa cells, weakly in LNCaP and PC3 prostate cancer cells and undetectable in 1321N1 astrocytoma cells. (B) Analysis of RBPMS2 transcript level in GIST882 cells upon incubation with 1 μM of Imatinib (inhibitor of KIT activity) or with DMSO alone (control DMSO) for 6, 24 and 48 h. RBPMS2 transcript level is not influenced by inhibition of KIT activity. Control, untreated cells. (C) Analysis of RBPMS2 transcript level in GIST881 cells following incubation with 5 μM of U0126 (inhibitor of MEK activity) or with DMSO alone (control DMSO) for 6, 24 and 48 h. RBPMS2 transcript level is not influenced by MEK inhibition. Control, untreated cells.

FIG. 3: The requirement of the dimerization of RBPMS2 to induce the dedifferenciation of visceral smooth muscle cell

Quantification of mitotic cells using anti-PH3 antibodies in primary cultured SMCs infected with RCAS-empty, RCAS-RBPMS2 and RCAS-RBPMS2 L49E retroviruses for 7 days.

### EXAMPLES

### Example 1

### High Expression of the RNA-Binding Protein RBPMS2 in Gastrointestinal Stromal Tumors (Hapkova et al., 2013)

Material & Methods

Tumor Samples and KIT and PDGFRA Mutational Analysis

Paraffin-embedded tumor samples from primary GISTs of 23 adult patients before Imatinib treatment were collected in the Department of Clinical and Molecular Pathology of the Olomouc University Hospital (Olomouc, Czech Republic). The risk of clinically aggressive behavior was evaluated according to the consensus approach published by Fletcher and coworkers (Fletcher et al., 2002). Control samples were normal gastrointestinal tissue specimens isolated from adult epithelial-derived tumors. For KIT and PDGFRA mutational analysis, genomic DNA was extracted from the paraffin-embedded GIST samples using the QIAamp DNA FFPE Tissue Kit (Qiagen). Specific PCR primers were used to analyze KIT exons 9, 11, 13 and 17 and PDGFRA exons 12, 13, 17 and 18 as previously described (Willmore-Payne et al., 2005). The GIST tissue microarray (DAA1, SuperBioChips Laboratories) contained 48 GIST and 9 normal adult gastrointestinal tissue samples. The microarray GIST samples were KIT-positive by immunodetection, but their KIT mutation status was unknown.

RNA Isolation and Quantitative RT-PCR

Total RNA was extracted from paraffin-embedded GIST samples using the High Pure RNA Paraffin kit (Roche Diagnostic). For quantitative RT-PCR analysis, gene expression levels were measured using the LightCycler technology (Roche Diagnostics). KIT, TMEMJ6A, RBPMS2, PDGFRA, HPRT, Calponin, αSMA, Desmin, SM22 and PCNA PCR primers were designed using the LightCycler Probe Design software 2.0. Each sample was assayed in three independent experiments in triplicate. Expression levels were determined with the LightCycler analysis software (version 3.5) relative to standard curves. Data were represented as the mean level of gene expression relative to the expression of the reference gene HPRT (Roche Diagnostic). For cell culture experiments, RNA isolation was done with the RNeasy Mini kit (Qiagen) and quantitative RT-PCR was performed using Power Sybergreen (Applied biosystems). Data were represented as the mean level of gene expression relative to the expression of the reference genes ABL, TTC1 and UBC for the comparison of RBPMS2 expression between different cell lines and GAPDH and ACTIN for GIST882 cells, which were treated with either Imatinib or U0126.

Production of Anti-RBPMS2 Antibodies and Cell Culture

The anti-human RBPMS2 rabbit polyclonal antibody was raised using a synthetic peptide corresponding to the C-terminus (amino acids 195-207) of human RBPMS2 (amino acids 1-209; Accession number NM_{—}194272, NP_{—}919248). Anti-RBPMS2 antibodies were purified using protein A-sepharose and were tested by ELISA (Biotem, France). The human GIST cell line GIST882 was maintained in DMEM (GIBCO) supplemented with 10% Fetal Bovine Serum (FBS), 2% Penicilline-Streptomycin (Lonza) and incubated with 1 μM of Imatinib (inhibitor of KIT activity) (LC Laboratory) or 5 μM of U0126 (MEK inhibitor) (Sigma-Aldrich), as previously described (Gromova et al., 2011). The human Embryonic Kidney 293 (HEK293) cell line was grown in DMEM supplemented with 10% FBS and transfected, using JetPEi™ (Polyplus, France), with 5 μg of a construct in which the full length human RBPMS2 cDNA was subcloned in the pCS2 vector with an in frame N-terminal HA tag and the CMV promoter. Cells were analyzed after 24 h. For western blot analyses, cells were lysed and protein extracts (20 μg) were separated on 10% polyacrylamide gels (Bio-Rad Laboratories), transferred onto nitrocellulose membranes (Amersham Hybond-ECL) and incubated with anti-RBPMS2 (homemade), anti-Tubulin (Abeam) and anti-HA (Santa Cruz Biotechnologies) primary antibodies overnight. After several washes, membranes were incubated with the relevant horseradish peroxidase-conjugated secondary antibodies (Perkin Elmer). Detection was performed by chemiluminescence (Santa Cruz Biotechnologies) on Kodak films. Tubulin expression served as a loading control.

Immunohistochemistry and In Situ Hybridization

Immunofluorescence and immunohistochemistry analysis of paraffin-embedded GIST sections were performed as described (Rouleau et al., 2009; Rouleau et al., 2012). Briefly, sections were deparaffinized with Histoclear (VWR, France) and rehydrated through a series of graded alcohols to PBS. For immunodetection, heatinduced antigen retrieval was carried out in 10 mM sodium citrate solution (pH9.0). Endogenous peroxidases were inactivated by incubation in 3% H2O2 (Sigma-Aldrich) for 30 minutes. Anti-RBPMS2 (home-made), -Ki67 (NeoMarker), -αSMA (Sigma-Aldrich), -CD34 (Clinisciences), -S100 (Clinisciences), -KIT (Zymed), -Desmin (Euromedex) and -TMEM16A (also called DOG1) (Abeam) primary antibodies were used. Specific mouse or rabbit anti-IgG biotinylated secondary antibodies were used with the avidin-peroxidase reagent (Vector) and antibody reactions were detected with 3,3′-Diaminobenzidine (Sigma-Aldrich). As control, each GIST sections were tested without primary antibodies. Hematoxylin and Eosin (H&E) staining was performed according to standard procedures. In situ hybridization experiments using paraffin sections were carried out as described (Come et al., 2006; Notarnicola et al., 2012). Anti-sense riboprobes were generated by PCR amplification of human RBPMS2 cDNA using specific primer sets and subcloned in pGEM T Easy Vector (Promega, France) as previously published (Notarnicola et al., 2012). Images were acquired using a Nikon-AZ100 stereomicroscope and a Carl-Zeiss Axiolmager microscope.

Statistical Analysis

Statistical analysis was carried out using the Mann-Whitney test as previously described (Notarnicola et al., 2012).

Results

RBPMS2 Transcripts are Detected in Adult GISTs

To determine whether the RNA-binding protein RBPMS2 was expressed in GISTs, the inventors first analyzed by in situ hybridization a commercial tissue microarray that included 48 KIT-positive GISTs (from low to high risk) and 9 normal adult gastrointestinal tissues. The level of RBPMS2 transcripts was very low in smooth muscles of normal adult gastrointestinal tissues. Conversely, RBPMS2 was strongly expressed in 36 of the 48 GIST samples (75%), independently of their localization and the risk of aggressive behavior.

Clinicopathological Features of the GIST Cohort

Due to the limited clinical data and the absence of information on the KIT and PDGFRA mutational status for the GISTs included in the microarray and in order to better characterize RBPMS2 expression in GISTs, the inventors analyzed GIST samples from a cohort of patients from Olomouc University Hospital. The group included 16 males and 7 females (male/female ratio: 1.44) with a mean age of 63.4 years (range: 36 to 81 years) who had exclusively primary GISTs located in the esophagus (n=4), stomach (n=5), small intestine (n=8), rectum (n=3), abdomen (n=2) and soft tissues (n=1) (Table 1). All GISTs included in this study were characterized by using classical histopathological and immunohistochemical approaches with anti-KIT, -SMA, -CD34, -Desmin, -S100 and -TMEM16A (DOG-1) antibodies (Table 2). GISTs were divided in spindle (n=11), epithelioid (n=4) and mixed cell (epithelioid and spindle) (n=8) tumors based on the cell morphology (Table 1). Mitotic cells were detected with the anti-Ki67 antibody (summary in Table 2) and tumors were classified as low risk (n=10) or high risk (n=13) as previously described (Miettinen and Lasota, 2006). KIT and PDGFRA mutational analysis was available for 21 GISTs and showed that 10 tumors (47.6%) had KIT mutations (three GISTs with KIT mutations in exon 9, six tumors with mutations in exon 11 and one tumor with a mutation in exon 13), four (19%) had a PDGFRA mutation (one missense and two silent mutations in exon 18 and one missense mutation in exon 12) and seven (33.4%) had wild type (WT) KIT and PDGFRA (Table 1). Quantitative RT-PCR analysis of KIT and PDGFRA expression levels indicated, as previously published, that in all the analyzed GIST samples (n=20) KIT and PDGFRA were up-regulated in comparison to normal gastrointestinal samples of adult colon, stomach and small intestine.

TABLE 1
Summary of the clinical and morphological features, risk assessment, KIT and PDGFRA
mutational status as well as KIT and RBPMS2 expressions in the GIST cohort.

| Pa- | | Age | | | | | KIT or | KIT | RBPMS2 | RBPMS2 |
|---|---|---|---|---|---|---|---|---|---|---|
| tient | Sex | (y) | Site | Morphology | Size (mm) | Risk | PDGFRA mutations | RT-PCR | RT-PCR | IHC |
| 1 | M | 73 | Small intestine | Spindle | 90 × 100 × 110 | High Risk | Exon 9 ins AY502-503 KIT | +++ | +++ | ++ |
| 2 | M | 73 | Soft tissue | Epithelioid | 12 × 5 × 12 | High Risk | Exon 9 ins AY502-503 KIT | +++ | +++ | +++ |
| 3 | M | 74 | Duodenum | Spindle | 70 × 75 × 85 | High Risk | Exon 9 ins AY502-503 KIT | ++ | ++ | +++ |
| 4 | M | 71 | Small intestine | Spindle | 14 × 5 × 14 | Low Risk | Exon 11 V559D KIT | ++ | +++ | +++ |
| 5 | M | 60 | Rectum | Spindle | 35 × 20 × 15 | Low Risk | Exon 11 V559D KIT | − | ++ | ++ |
| 6 | M | 71 | Stomach | Mixed | 15 × 12 × 10 | High Risk | Exon 11 del/ins KIT | +++ | +++ | + |
| 7 | M | 77 | Esophagus | Spindle | 22 × 18 × 40 | High Risk | Exon 11 del/ins KIT | +++ | +++ | ++ |
| 8 | M | 74 | Rectum | Spindle | 80 × 75 × 60 | Low Risk | Exon 11 del/ins KIT | + | +++ | ++ |
| 9 | M | 66 | Stomach | Spindle | 9 × 5 × 5 | Low Risk | Exon 13 K642E KIT | ++ | +++ | NA |
| 10 | F | 67 | Stomach | Epithelioid | 43 × 47 × 35 | High Risk | E12 V561D PDGFRA | + | ++ | ++ |
| 11 | F | 62 | Stomach | Mixed | NA | Low Risk | Exon 18 D842Y PDGFRA | ++ | +++ | NA |
| 12 | F | 58 | Colon | Mixed | 10 × 10 × 10 | Low Risk | Exon 18 silent mutation | ++ | ++ | ++ |
| | | | | | | | V824V PDGFRA | | | |
| 13 | M | 36 | Recto-peritoneum | Mixed | 65 × 45 × 42 | Low Risk | Exon 18 silent mutation | + | ++ | + |
| | | | | | | | V824V PDGFRA | | | |
| 14 | F | 70 | Esophagus | Mixed | 50 × 30 × 30 | Low Risk | WT | +++ | +++ | ++ |
| 15 | M | 48 | Esophagus | Spindle | 35 × 25 × 20 | High Risk | WT | + | + | ++ |
| 16 | M | 61 | Stomach | Spindle | 45 × 25 × 10 | Low Risk | WT | ++ | +++ | NA |
| 17 | M | 42 | Small intestine | Muted | 40 × 30 × 20 | High Risk | WT | +++ | +++ | + |
| 18 | M | 60 | Duodenum | Mixed | 40 × 32 × 22 | Low Risk | WT | ++ | +++ | + |
| 19 | M | 81 | Rectum | Mixed | 1 × 3 × 15 | High Risk | WT | + | ++ | ++ |
| 20 | F | 55 | Recto-peritoneum | Epithelioid | 90 × 70 × 50 | High Risk | WT | +++ | +++ | ++ |
| 21 | M | 65 | Small intestine | Epithelioid | 12 × 8 × 8 | High Risk | NA | NA | NA | ++ |
| 22 | F | 55 | Small intestine | Spindle | 150 × 13 × 110 | High Risk | NA | NA | NA | + |
| 23 | F | 60 | Stomach | Spindle | 50 × 40 × 50 | High Risk | Exon 11 del/ins KIT | NA | NA | + |

TABLE 2
Results of immunohistochemical staining
for KIT (CD117), TMEM16A, CD34,
αSMA (Smooth Muscle Actin), DESMIN and S100
(n = 23 GIST samples) in adult GIST cohort.

| | Positive, | Negative, | NA | Total, |
|---|---|---|---|---|
| Immunohistochemistry | n (%) | n (%) | (%) | n |
| KIT (CD117) | 22 (96) | 1 (4) | 0 (0) | 23 |
| TMEM16A | 16 (69.5) | 4 (17.5) | 3 (13) | 23 |
| CD34 | 15 (65) | 4 (17.5) | 4 (17.5) | 23 |
| SMOOTH MUSCLE | 9 (39) | 8 (35) | 6 (26) | 23 |
| ACTIN | | | | |
| DESMIN | 2 (9) | 12 (52) | 9 (39) | 23 |
| S100 | 7 (30) | 10 (44) | 6 (26) | 23 |

RBPMS2 is Strongly Expressed in Malignant GISTs

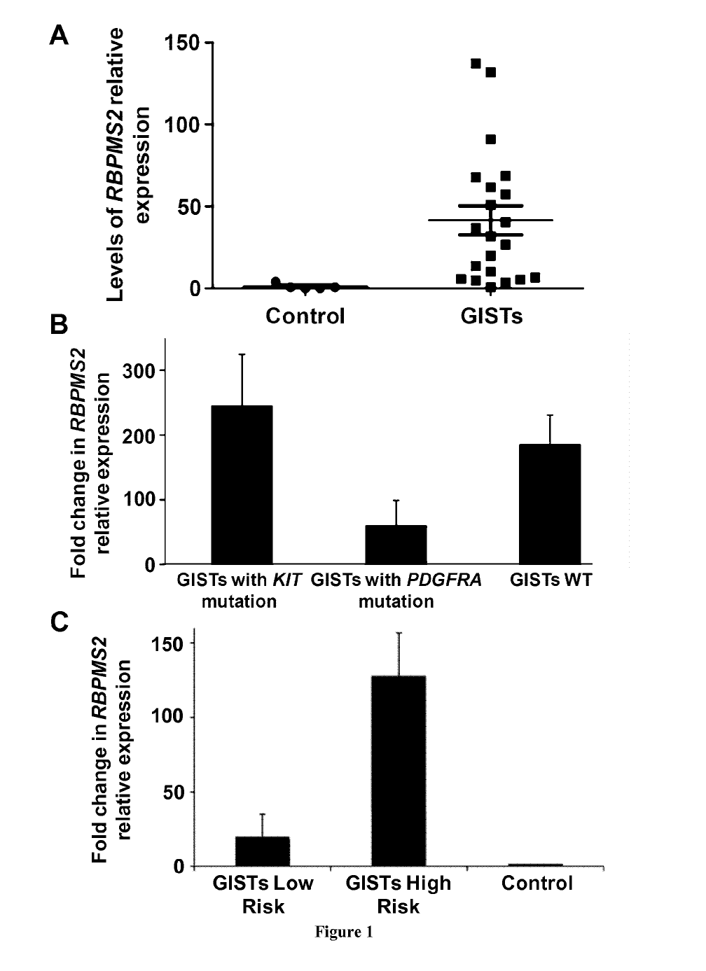

The inventors then analyzed the levels of RBPMS2 transcripts in this GIST cohort and in gastrointestinal control samples by quantitative RT-PCR. In most of the analyzed GIST samples (19 of 20) RBPMS2 expression level was higher than in control samples (Table 1) and the mean RBPMS2 level in GISTs was 42-fold higher than in control samples (p<0.001) (Figure A). The level of KIT and PDGFRA did not significantly correlated with the amount of RBPMS2 expression in the tumors. Conversely, RBPMS2 expression was higher in GISTs harboring KIT mutations than in tumors with wild type KIT and with wild type KIT or PDGFRA mutations (FIG. 1B). GISTs with PDGFRA mutations were characterized by the lowest RBPMS2 expression levels (FIG. 1B). Finally, RBPMS2 levels were 64-fold higher in GIST samples with high risk of aggressive behavior than in adult control digestive samples (FIG. 1C) and 6.2-fold higher in high risk than in low risk GISTs (FIG. 1C).

RBPMS2 Protein is Highly Expressed in Adult GISTs

In order to examine RBPMS2 protein expression, the inventors generated antibodies directed against the C-terminus of human RBPMS2 (amino acids 195-207). The efficiency and the specificity of these anti-RBPMS2 antibodies were confirmed by western blot and immunofluorescence analyses in HEK293 cells that express HA tagged RBPMS2. In western blots, the anti-RBPMS2 antibody identified a single band of 27 kDa, corresponding to the predicted size of human RBPMS2 fused to the HA tag. In addition, this band was also detected with the anti-HA antibody. Moreover, the anti-RBPMS2- and -HA antibodies both detected an epitope localized in the cytoplasmic compartment of such cells. Finally, the RBPMS2 signal was specifically lost when the anti-RBPMS2 antibody was pre-incubated with the RBPMS2 C-terminal peptide used to produce the antibody. The inventors then examined the expression of RBPMS2 by immunohistochemistry in control gastrointestinal and GIST samples (Table 3). RBPMS2 was faintly but reproductively detected in the gastrointestinal musculature with the exception of the myenteric plexus. In contrast, it was strongly detected in 87% of the analyzed GIST samples (Table 3) and its expression level was comparable in spindle, epithelioid and mixed cell tumor variants.

TABLE 3
Summary of KIT (CD117) and RBPMS2 protein expression in the GIST
cohort (n = 23). NA, not available.

| Immunohistochemistry | Positive, n (%) | Negative, n (%) | NA (%) | Total, n |
|---|---|---|---|---|
| KIT (CD117) | 22 (96) | 1 (4) | 0 (0) | 23 |
| RBPMS2 | 20 (87) | 0 (0) | 3 (13) | 23 |

RBPMS2 Expression and Regulation in GIST882 Cells

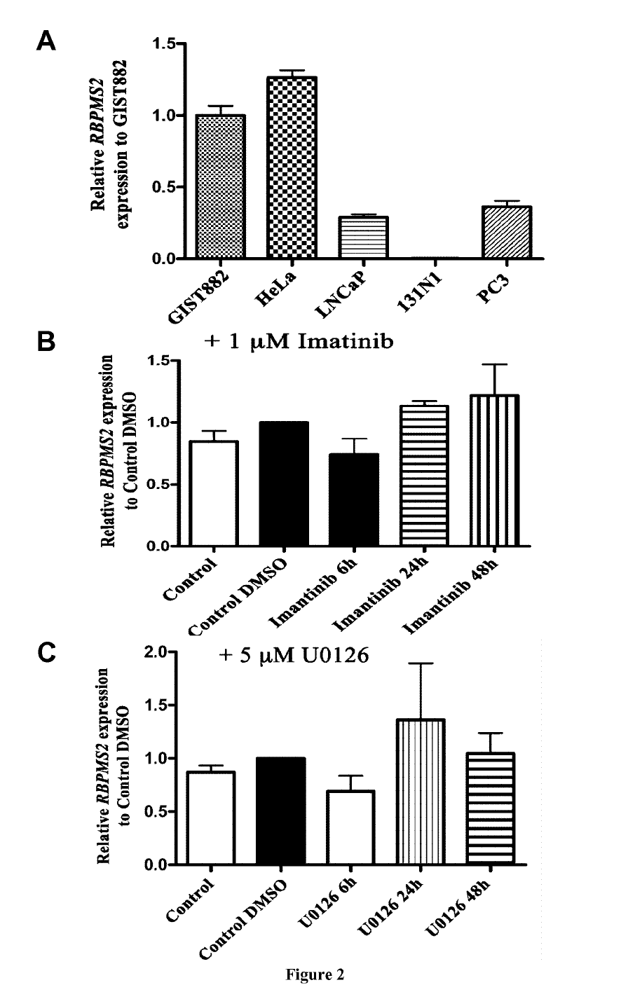

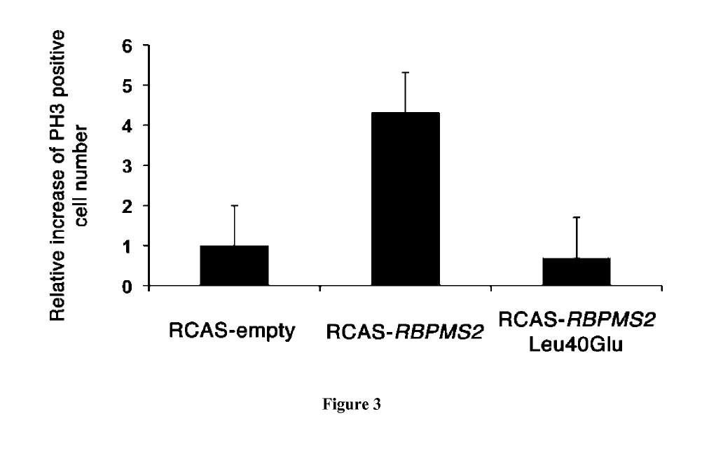

To position RBPMS2 in the KIT signaling pathway in GISTs, the inventors first compared RBPMS2 expression in GIST882 (a GIST cell line homozygous for the oncogenic KIT mutation K641E with strong KIT expression and high level of KIT activity) (Tuveson et al., 2001), HeLa and 1321N1 (human astrocytoma) cells as well as in two prostate cancer cell lines (PC3 and LNCaP). RBPMS2 mRNA level was relatively high in GIST882 and HeLa cells in comparison to 1321N1 cells and the two prostate cancer cell lines (FIG. 2A). Then GIST882 cells were treated with 1 μM of Imatinib (a specific inhibitor of KIT activity) (Tuveson et al., 2001) or with 5 μM of U0126 (a selective inhibitor of MEK which is a downstream effector of the KIT signaling pathway) for 6, 24 and 48 h (FIGS. 2B and 2C) and RBPMS2 expression was determined by quantitative PCR. No significant changes in RBPMS2 mRNA levels were observed following addition of Imatinib or U0126 in the culture medium (FIGS. 2B and 2C).

Discussion

In this study the inventors analyzed the expression of the RNA-binding protein RBPMS2 in a cohort of GIST samples that were classified according to their KIT/PDGFRA mutational status, risk of aggressive behavior and histological pattern (spindle, epithelioid and mixed cell phenotype).

The inventors found that RBPMS2 mRNA and protein expression was significantly higher in GIST samples than in control gastrointestinal tissues, particularly in high risk tumors. The levels of KIT and PDGFRA did not significantly correlate with the amount of RBPMS2 expression. However, RBPMS2 mRNA levels were higher in GISTs harboring KIT mutations than in tumors with PDGRA mutations or with wild type KIT and PDGFRA. This difference could be due to the different origins of KIT and PDGFRA expressing cells and would suggest that RBPMS2 and KIT expression are linked. However, in the GIST882 cell line that carries the KIT mutation K641E and shows high KIT activity the inventors did not observe a correlation between RBPMS2 expression and KIT activity. In contrast, using primary digestive smooth muscle cultures the inventors found that RBPMS2 over-expression induces a 2-fold increase of KIT mRNA level.

Chromosomal alterations during GIST progression have been described and could be involved in GIST prognosis (Corless et al., 2011). Specifically, losses and gains on chromosome 15 between 15q22.1 and 15q25.3 have been clearly correlated with poor outcome (Ylipää et al., 2010). As the RBPMS2 gene is localized on chromosome 15q22.31, the inventors can hypothesize based on our results that elimination of a negative regulatory sequence necessary for correct RBPMS2 expression could alter the endogenous expression of RBPMS2, thus favoring dedifferentiation of mesenchymal cells that will give rise to GIST tumors. A through characterization of the correlation between alterations of this genome region and RBPMS2 expression is now required.

In conclusion, the present invention show that most of the analyzed GISTs are characterized by abnormally elevated expression of RBPMS2, demonstrating that RBPMS2 present an indicator for tumor progression and a target for cancer therapy in GIST.

### Example 2

Material & Methods

Plasmids

The human RBPMS2 cDNA sequence coding to the aminoacid 27 to 117 was subcloned into pET22 (pET22-human-RBPMS2-Nter). Substitution of Leucine by Glutamic Acid in position 49 of the human RBPMS2 sequence (L49E) was introduced by QuikChange site-directed mutagenesis method (Stratagene) in order to create pET22-human-RBPMS2-Nter L49E plasmid. The full-length human RBPMS2 cDNA was subcloned in the pCS2 vector with an in frame N-terminal HA tag and the CMV promoter (pCS2-HA-human-RBPMS2). The full-length human RBPMS2 and RBPMS2 L49E were subcloned in the pHRTK vector with an in frame N-terminal Myc tag and the CMV promoter (respectively pHRTK-Myc-human-RBPMS2 and pHRTK-Myc-human-RBPMS2 L49E). HA-tagged human TC10 was previously described (Coisy-Quivy et al., 2009). Myc-tagged chick full-length RBPMS2 with corresponding Leu40Glu substitution was cloned into the RCAS vector to produce replication-competent retroviruses that express Myc-RBPMS2 Leu40Glu. Myc-tagged chick full-length RBPMS2 (RCAS-Myc-gallus-RBPMS2), GFP (RCAS-GFP) and Myc-NICD were previously described (Notarnicola et al., 2012; Moniot et al., 2004; Shih and Holland, 2006). All plasmids were checked by DNA sequencing and protein expression.

DuoLink Analysis

For DuoLink in situ Proximity Ligation Assay (PLA) (adapted from Soderberg et al.), DF1 cells transfected with different plasmid combination were labeled with mouse anti-HA (Santa Cruz Biotechnologies) and rabbit anti-Myc (Ozyme) primary antibodies and incubated with a pair of nucleotide-labeled secondary antibodies (rabbit PLA probe MINUS and mouse PLA probe PLUS; OLINK Biosciences, Uppsala Sweden) in hybridization solution. In addition, secondary mouse and rabbit anti-IgG respectively coupled to Alexa 488 and 555 were incubated to detect protein expression. Interactions between the PLA probes, possible when within a distance less than 40 nm, were revealed by adding a ligase and by amplification of a rolling-circle product using far red labeled oligonucleotides and a polymerase, according to the manufacturer's instructions. Cells were counterstained using Duolink II Mounting Medium with 4′,6-Di-Amidino-2-Phenyl-Indole. Signals indicative of interactions were detected by confocal microscopy as fluorescent dots.

Recombinant Proteins

pET22-human-RBPMS2-Nter and pET22-human-RBPMS2-Nter L49E plasmids were transformed into Escherichia coli strain BL21ADE3 for protein overexpression using T7 RNA polymerase. Proteins were purified as previously described (Yang et al., 2009).

NMR Spectroscopy

All NMR experiments were carried out at 27° C. on a Bruker Avance III 700 spectrometer equipped with 5 mm z-gradient TCI cryoprobe, using the standard pulse sequences (Sattler et al., 1999). NMR samples consist on approximately 0.5 mM ¹⁵N- or ¹⁵N,¹³C-labeled protein dissolved in 10 mM acetate buffer, 50 mM NaCl, pH 4.6 with 5% D2O for the lock. ¹H chemical shifts were directly referenced to the methyl resonance of DSS, while ¹³C and ¹⁵N chemical shifts were referenced indirectly to the absolute ¹⁵N/¹H or ¹³C/¹H frequency ratios. All NMR spectra were processed and analysed with GIFA (Pons et al., 1996). Structures were validated using PROCHECK (Laskowski et al., 1993).

Fluorescence Anisotropy

Human RBPMS2-Nter and RBPMS2-Nter L49E were labelled with the NHS ester of ATTO647N in 20 mM Na-phosphate buffer pH 7.5 with 50 mM KCl during 3 hours at room temperature. Labelled proteins were separated from the free dye using a 2 ml Zeba spin desalting column (Thermo Scientific) equilibrated in binding buffer (20 mM Tris-HCl pH 7.5, 100 mM KCl). Anisotropy measurements were carried out at 25° C. in dilution mode. RBPMS2-Nter-ATT03457N and RBPMS2-Nter L49E-ATTO647N (2 nM final) were then mixed with different RNAs (2 μM final) in binding buffer. The mixture was serially diluted with the same buffer containing only 2 nM RBPMS2-Nter-ATTO647N or RBPMS2-Nter L49E-ATTO647N. Measurements were made at each dilution in Corning black 384 wells assay plate with a TECAN Safire2 in polarization mode.

Using the pCMV6-XL5 plasmid that contains the human NOGGIN cDNA including the 5′ and 3′ untranslated regions and the open reading frame (OriGene), different DNA matrix were constructed by PCR with forward primers that carried T7 promoter sequence. These amplified DNA were used as template to synthesize in vitro human NOGGIN RNA using T7 high yield RNA synthesis Kit (New England Biolabs). Following sequences were used: 214-1447, 214-838, 214-538, 518-838, 818-1447.

Immunoprecipitation

The avian DF-1 chicken fibroblast cell line (ATCC-LGC) was grown in DMEM supplemented with 10% FBS and transfected using Lipofectamine 2000 (Invitrogen, France) with above described constructs. Cells were analyzed after 24 h. Cells were lysed using Lysis Buffer (20 mM Tris pH8, 50 mM NaCl, 1% NP40, cOmplete, EDTA-free Protease Inhibitor Cocktail (Roche)). 50 mg of total DF1 protein lysates were incubated directly with the rabbit anti-Myc antibodies (Ozyme) pre-adsorbed to protein A-Sepharose CL-4B (GE Healthcare) for 1 h at 4° C. in Immunoprecipitation Buffer (50 mM Tris pH8, 150 mM NaCl, 0.4% NP40, cOmplete, EDTA-free Protease Inhibitor Cocktail (Roche)). After extensive washing, bound proteins were eluted by boiling in SDS-PAGE sample buffer, analyzed by 12% SDS-PAGE, and transferred to nitrocellulose. The membrane was blocked with 10% nonfat milk in TBS+0.1% Tween and probed with mouse anti-HA or rabbit anti-Myc polyclonal antibodies overnight. After several washes, membranes were incubated with the relevant horseradish peroxidase-conjugated secondary antibodies (Perkin Elmer). Detection was performed by chemiluminescence (Santa Cruz Biotechnologies) on Kodak films.

Avian Retroviral Misexpression System

Fertilized White Leghorn eggs from Haas Farm (France) were incubated at 38° C. in humidified incubators. Gastrointestinal tissues from chick embryos were dissected as described (Moniot et al., 2004). Retroviral constructs were transfected into the DF-1 chicken fibroblast cell line (ATCC-LGC) to produce retroviruses. Retroviruses were injected into the splanchnopleural mesoderm of Stage-10 chicken embryos to target the stomach mesenchyme (Moniot et al., 2004; Notarnicola et al., 2012). Eggs were then placed at 38° C. until harvested.

Primary Cultured SMCs and Analysis

Primary cultures from E15 gizzard muscle were prepared as described (Notarnicola et al., 2012). Briefly, the tunica muscularis was carefully separated from the serosa and tunica mucosa before collagenase dissociation. Isolated cells were cultured in Dulbecco's modified Eagle medium (DMEM) in presence of 0.2% BSA and 5 μg/ml insulin in Matrigel-coated plates to maintain the cell differentiation status (more than 95% of isolated cells were Desmin- and αSMA-positive, data not shown). Differentiated SMCs were then infected with RCAS-empty, or RCAS-Myc-gallus-RBPMS2, or RCAS-Myc-gallus-RBPM S2 L40E retroviruses and maintained in culture for 3 days. In our conditions, avian retroviruses have a high tropism to infect SMCs. For immunodetection, anti-Myc (Ozyme), anti-avian Calponin (Sigma-Aldrich), and anti-Phospho-Histone H3-Ser10 (Millipore) antibodies were used. Nuclei were stained with Hoechst (Molecular Probes).

Immunofluorescence and In Situ Hybridization on Chick Stomach

Immunofluorescence experiments with chick paraffin-embedded sections and in situ hybridization experiments using dissected gut were carried out as described (Moniot et al., 2004; Notarnicola et al., 2012). For immunodetection, anti-Myc (Ozyme), and anti-avian Calponin (Sigma-Aldrich) antibodies were used. Nuclei were stained with Hoechst (Molecular Probes). The chick Noggin template was used (Notarnicola et al., 2012). Anti-sense Noggin riboprobes was generated by reverse transcription using chick Noggin template with incorporation of digoxigenin-UTP (Roche). Anti-digoxigenin antibodies coupled to alkaline phosphatase (Roche) were used to detect Noggin sens/antisens complexes with BM Purple solution (Roche). Images were acquired using and a Carl-Zeiss Axiolmager microscope (for immunofluorescence) and a Nikon-AZ100 stereomicroscope (for whole-mount in situ hybridization).

Results

In order to determinate the structure of the human RBPMS2 protein, the inventors looked at the structural organization of RBPMS2 by molecular modeling using the server @TOME-2 (Pons and Labesse, 2009). The inventors found that the N-terminus part of the human RBPMS2 protein (residues 27-117) was predicted to be structured as a RRM domain. Based on this result, the inventors have produced this domain in bacteria (RBPMS2-Nter) and purified it. Using NMR experiments, the inventors have confirmed the RRM fold for the N-terminus part of RBPMS2 protein, and found that this domain is exclusively present in the homodimeric form in solution.

To examine RBPMS2 homodimerization in vitro, the inventors conduct coImmunoprecipitation assays (coIP) using DF1 cell lysates expressing Myc- or HA-tagged RBPMS2 proteins and anti-Myc antibodies. The inventors observe that HA-tagged RBPMS2 coprecipitates with Myc-tagged RBPMS2. To test whether the interaction is specific or mediated by bridging RNA, the inventors performed coIP from RNase-treated assays and observe a specific homodimerization between RBPMS2 proteins. To confirm our results, the inventors include the small GTPase TC10 protein fused to HA tag as an additional negative control and observe no interaction with RBPMS2.

The inventors also investigate RBPMS2 homodimerization in cell culture using DuoLink technology, an in situ proximity ligation assay (PLA) that detects two proteins only when they are in close proximity. The inventors find that HA-RBPMS2 interacts with Myc-RBPMS2 in DF1 cells expressing both Myc- and HA-RBPMS2 proteins. For negative control, the inventors also test the interaction of RBPMS2 with unrelated Myc- or HA-tagged proteins, but we do not observe interaction. These data support that RBPMS2 is present as a homodimer in vivo.

Using NMR experiments done on human RBPMS2-Nter protein, the inventors found that homodimerization of RBPMS2 protein could involve the interaction of both Leucine 40 from each RBPMS2 protein. This Leucine 49 is conserved into all RBPMS2 homologs (the presence of the similar Leucine at position 40 in gallus). In order to test the hypothesis that Leucine 49 is involved in the dimerization complex of RBPMS2, the inventors substitute Leucine 49 by Glutamic Acid (L49E) that is predicted to avoid the dimerization process without alteration of global structure. The inventors analyze by NMR experiment their structure and observe that RBPMS2-Nter L49E is essentially present as a monomeric form in solution. The inventors previously showed that RBPMS2 is a RNA-Binding Protein that can bind RNAs via its RRM domain and using immunoprecipitation of tagged avian RBPMS2 protein we found that Noggin mRNA and RBPMS2 are present in a common RNA-protein complex (Notarnicola et al., 2012). In order to analyze the impact of L49E substitution of RBPMS2, the inventors evaluate the capacity of human purified RBPMS2-Nter protein that contains the RRM domain to bind to the human NOGGIN mRNA synthesised in vitro by fluorescence anisotropy-based binding assays. The inventors find that RBPMS2-Nter binds to human NOGGIN mRNA and identify that sequence between 518 to 838 is involved in this binding. The inventors also find that human RBPMS-Nter L49E binds to similar NOGGIN sequence without affinity difference, suggesting that L49E substitution did neither alter its capacity to bind RNA nor its structure. These data support that L49E substitution in RBPMS2 protein does not alter the structure of RBPMS2 protein nor its ability to bind NOGGIN RNA, but conducts to the presence of monomeric RBPMS2 protein in solution.

In addition, the inventors conduct coIP using DF1 cell lysates expressing HA-tagged RBPMS2 or Myc-RBPMS2 or Myc-RBPMS2 L40E and anti-Myc antibodies. The inventors observe that HA-tagged RBPMS2 coprecipitates with Myc-tagged RBPMS2 but faintly with Myc-tagged RBPMS2 L40E. The Leucine to Glutamic Acid substitution abrogates 83.5% of the dimerization. The inventors also test with DuoLink technique the impact of Leucine to Glutamic Acid substitution in cell culture. The inventors find that HA-RBPMS2 does not interact with Myc-RBPMS2 L40E in DF1 cells expressing both Myc- and HA-RBPMS2 proteins. These data demonstrate that substitution of Leucine 49 to Glutamic acid of RBPMS2 protein avoid RBPMS2 dimerization in vitro and in vivo.

To test the function or the requirement of the RBPMS2 dimerization in cellulo, the inventors analyze the impact of RBPMS2 L40E in primary SMC cell culture and compare it to the action of RBPMS2. The inventors establish primary cultures on Matrigel of visceral differentiated SMCs from E15 gizzard muscles in serum-free medium supplemented with insulin (Notarnicola et al., 2012). Control primary cultured SMCs in the presence of replication-competent retroviruses without transgene (RCAS-empty) were spindle-shaped and homogenously expressed αSMA and calponin, 2 SMC contractile markers, in highly organized filament bundles. SMCs then were infected with replication-competent retroviruses (RCAS-RBPMS2 or RCAS-RBPMS2 L40E construct or RCAS-empty [control]) for 3 days. Although in control cells the expression of calponin remained unchanged, in SMCs infected with Myc-tagged RBPMS2 calponin expression is lost. The inventors also observe that SMCs infected with Myc-tagged RBPMS2 L40E calponin expression remain unchanged. The inventors also investigate the impact of RBPMS2 L40E on primary cultured SMCs with the analysis of the expression of phosphorylated Histone 3-Ser10 (PH3), a standard marker of G2/M transition. After 6 days of culture, the number of PH3-positive cells was 4.5-fold higher in cells that over-expressed RBPMS2 than in controls (FIG. 3). In addition, the inventors observe no change in the number of PH3-positive cells in cells that over-expressed RBPMS2 L40E compare to the controls, demonstrating that RBPMS2 dimerization is essential to induce the dedifferentiation of the SMCs.

To test the function or the requirement of the RBPMS2 dimerization in vivo, the inventors analyze the impact of RBPMS2 L40E and RBPMS2 during the development of the avian gastrointestinal tract and compare it to the action of RBPMS2. The inventors use the avian replication-competent retroviral misexpression system that allows in vivo targeting of specific genes in the stomach mesenchyme and the sustained expression of transgene throughout visceral muscle development and differentiation. As previously demonstrated (Notarnicola et al., 2012), sustained RBPMS2 expression results in a dramatic alteration of the stomach morphology. Specifically, the proventriculus, which is the glandular part of the chick stomach was hypertrophied, whereas the gizzard was denser and malformed in comparison with controls that overexpressed GFP alone. In contrast, to the action of RBPMS2, the sustained expression of RBPMS2 L40E does not induce any morphological change of the infected stomachs (n=26), stomachs presenting expression of transgenes.

The inventors previously showed that sustained RBPMS2 expression in the GI tract induces the upregulation of Noggin mRNAs in vivo (Notarnicola et al., 2012). Using the avian retroviral misexpression technique, the inventors analyze the impact of RBPMS2 L49E, RBPMS2 and GFP as control on Noggin mRNA expression by in situ hybridization. The inventors observe that RBPMS2 misexpression in the gastrointestinal mesenchyme is always associated to the upregulation of Noggin mRNA in infected stomach in comparison to controls. In contrast, the inventors find that RBPMS2 L49E misexpression does not induce any up-regulation of Noggin mRNA (n=17, infected stomachs analyzed by in situ hybridization), demonstrating that RBPMS2 dimerization is essential to induce upregulation of Noggin mRNA.

The inventors show that the conserved RBPMS2 protein, homodimerizes via its RRM domain and that this interaction is essential for its function. The inventors also demonstrate that the newly identified RRM-homodimerization motif (residues 47-50 of the SEQ ID NO:1) is crucial for the function of RBPMS2 at the cell and tissue levels.

### REFERENCES

Throughout this application, various references describe the state of the art to which this invention pertains. The disclosures of these references are hereby incorporated by reference into the present disclosure.
Come, C., Magnino, F., Bibeau, F., et al., 2006. Snail and slug play distinct roles during breast carcinoma progression. Clin. Cancer Res. 12, 5395-5402.
Corless, C. L., Barnett, C. M., Heinrich, M. C., 2011. Gastrointestinal stromal tumours: origin and molecular oncology. Nat. Rev. Cancer. 11, 865-878.
Delahaye, N. F., Rusakiewicz, S., Martins, I., et al., 2011. Alternatively spliced NKp30 isoforms affect the prognosis of gastrointestinal stromal tumors. Nat Med. 17, 700-707.
Faure S., de Santa Barbara, P. 2011. Molecular embryology of the foregut. J. Pediatr. Gastroenterol. Nutr. 52, S2-3.
Fletcher, C. D., Berman, J. J., Corless, C., et al., 2002. Diagnosis of gastrointestinal stromal tumors: A consensus approach. Hum Pathol. 33, 459-465.
Gromova, P., Rubin, B. P., Thys. A., et al., 2011. Neurotensin receptor 1 is expressed in gastrointestinal stromal tumors but not in interstitial cells of Cajal. PlosOne. 6, e14710.
Hirota, S., Isozaki, K., Moriyama, Y., et al., 1998. Gain-of-function mutations of c-kit in human gastrointestinal stromal tumors. Science. 279, 577-580.
Joensuu, H., Roberts, P. J., Sarlomo-Rikala, M., et al., 2001. Effect of the tyrosine kinase inhibitor STI571 in a patient with a metastatic gastrointestinal stromal tumor. N. Engl. J. Med. 344, 1052-1056.
Le Guen, L., Notarnicola, C., de Santa Barbara, P. 2009. Intermuscular tendons are essential for the development of vertebrate stomach. Development. 136, 791-801.
Miettinen M., Lasota J. 2006. Gastrointestinal stromal tumors: review on morphology, molecular pathology, prognosis, and differential diagnosis. Arch Pathol Lab Med. 130, 1466-78.
Notarnicola, C., Rouleau, C., Le Guen, L., et al., 2012. The RNA binding protein RBPMS2 regulates gastrointestinal smooth muscle development. Gastroenterology. 143, 687-697.
Renouf, D. J., Wilson, L., Blanke, C. D. 2009. Successes and challenges in translational research: the development of targeted therapy for gastrointestinal stromal tumours. Clin. Cancer Res. 15, 3908-3911.
Rouleau, C., Matecki, S., Kalfa, N., et al., 2009. Activation of MAP kinase (ERK1/2) in human neonatal colonic enteric nervous system. Neurogastroenterol. Motil. 21, 207-214.
Rouleau, C., Rico, C., Hapkova, I., et al., 2012. Immunohistochemical analysis of bone morphological protein signaling pathway in human myometrium. Exp. Mol. Pathol. 93, 56-60.
Tuveson, D. A., Willis, N. A., Jacks, T., et al., 2001. STI571 inactivation of the gastrointestinal stromal tumor c-KIT oncoprotein: biological and clinical implications. Oncogene. 20, 5054-5058.
Sanders, K M, Koh, S D, Ward, S M., 2006. Interstitial cells of cajal as pacemakers in the gastrointestinal tract. Annu. Rev. Physiol. 68, 307-343.
St Johnston, D., 2005. Moving messages: the intracellular localization of mRNAs. Nat. Rev. Mol. Cell. Biol. 6, 363-375.
Xin, M., Small, E. M., Sutherland, L. B., et al., 2009. MicroRNAs miR-143 and miR-145 modulate cytoskeletal dynamics and responsiveness of smooth muscle cells to injury. Genes Dev. 23, 2166-2178.
Van Kouwenhove, M., Kedde, M., Agami, R. 2011. MicroRNA regulation by RNAbinding proteins and its implications for cancer. Nat. Rev. Cancer. 11, 644-656.
Willmore-Payne, C., Layfield L. J., Holden, J. A. 2005. C-KIT mutation analysis for diagnostic of gastrointestinal stromal tumors in fine needle aspiration specimens. Cancer. 105, 165-170.
Ylipää, A., Hunt, K. K., Yang, J., et al., 2010. Integrative genomic characterization and a genomic staging system for gastrointestinal stromal tumors. Cancer. 117, 380-389.
Coisy-Quivy M, Touzet O, Bourret A, Hipskind R A, Mercier J, Fort P, Philips A. TC10 controls human myofibril organization and is activated by the sarcomeric RhoGEF obscurin. J Cell Sci. 2009 Apr. 1; 122(Pt 7):947-56.
Laskowski R A, Moss D S, Thornton J M. 1993. Main-chain bond lengths and bond angles in protein structures. J. Mol. Biol. 231:1049-1067.
Moniot B, Biau S, Faure S, Nielsen C M, Berta P, Roberts D J, de Santa Barbara P. SOX9 specifies the pyloric sphincter epithelium through mesenchymal-epithelial signals. Development. 2004 August; 131(15):3795-804
Notarnicola C, Rouleau C, Le Guen L, Virsolvy A, Richard S, Faure S, De Santa Barbara P. The RNA-Binding Protein RBPMS2 Regulates Development of Gastrointestinal
Smooth Muscle. Gastroenterology. 2012 September; 143(3):687-697.e9
Pons J L, Labesse G. @TOME-2: a new pipeline for comparative modeling of protein-ligand complexes. Nucleic Acids Res. 2009 July; 37(Web Server issue):W485-91
Pons J L, Malliavin T E, Delsuc M A. 1996. Gifa V 4: A complete package for NMR data set processing. J. Biomol. NMR 8:445-452.
Sattler M, Schleucher J, Griesinger C. 1999. Heteronuclear multidimensional NMR experiments for the structure determination of proteins in solution employing pulsed field gradients. Prog. NMR Spectrosc. 34:93-158.
Shih A H, Holland E C. Notch signaling enhances nestin expression in gliomas.
Neoplasia. 2006 December; 8(12):1072-82.
Yang Y, Padilla A, Zhang C, Labesse G, Kaminski P A. Structural characterization of the mammalian deoxynucleotide N-hydrolase Rcl and its stabilizing interactions with two inhibitors. J Mol Biol. 2009 Dec. 4; 394(3):435-47.
Yang Y, Labesse G, Carrère-Kremer S, Esteves K, Kremer L, Cohen-Gonsaud M, Blanc-Potard A B. C-terminal domain of the virulence factor MgtC is a divergent ACT domain. J Bacteriol. 2012 Sep. 14.
Hapkova I, Skarda J, Rouleau C, Thys A, Notarnicola C, Janikova M, Bernex F, Rypka M, Vanderwinden J M, Faure S, Vesely J, de Santa Barbara P. High expression of the RNA-binding protein RBPMS2 in gastrointestinal stromal tumors. Exp Mol Pathol. 2013 April; 94(2):314-21.

1. A method of treating gastrointestinal stromal tumors in a subject in need thereof, comprising the step of administering to said subject a compound which is selected from the group consisting of RBPMS2 dimerization inhibitors, RBPMS2 antagonists and RBPMS2 expression inhibitors.

2. A method of preventing or treating high risk gastrointestinal stromal tumors in a subject in need thereof, comprising the step of administering to said subject a compound which is selected from the group consisting of RBPMS2 dimerization inhibitors, RBPMS2 antagonists and RBPMS2 expression inhibitors.

3. A method of screening a candidate compound for use as a drug for the treatment of gastrointestinal stromal tumors in a subject in need thereof, wherein the method comprises the steps of:
(i) providing a RBPMS2 or providing a cell, tissue sample or organism expressing the RBPMS2,
(ii) providing a candidate compound,
(iii) measuring the activity of the RBPMS2 in the presence of said candidate compound, and
(iv) selecting positively candidate compounds that block RBPMS2 dimerization, block the action of RBPMS2 or inhibit RBPMS2 expression.

4. A method of identifying a subject having a gastrointestinal stromal tumor which comprises the step of analyzing a biological sample from said subject by:
(i) determining an RBPMS2 expression level in the sample,
(ii) comparing the RBPMS2 expression level in the sample with a reference value,
(iii) detecting a differential in the RBPMS2 expression level between the sample and the reference value, wherein detection of a differential is indicative of a subject having a gastrointestinal stromal tumor.

5. A method of determining whether the gastrointestinal stromal tumor of a subject is a low risk tumor or a high risk tumor which comprises the step of analyzing a biological sample from said subject by:
(i) determining an RBPMS2 expression level in the sample,
(ii) comparing the RBPMS2 expression level in the sample with a reference value,
(iii) detecting a differential in the RBPMS2 expression level between the sample and the reference value, wherein detection of a differential is indicative that the gastrointestinal stromal tumor is a low risk tumor or a high risk tumor.

6. A method of preventing the progression of low risk gastrointestinal stromal tumors to high risk gastrointestinal stromal tumors in a subject in need thereof comprising the steps of:
i) determining whether the gastrointestinal stromal tumor of a subject is a low risk tumor or a high risk tumor by performing the method according to claim 5, and
ii) administering a compound which is selected from the group consisting of RBPMS2 dimerization inhibitors, RBPMS2 antagonists and RBPMS2 expression inhibitors if said subject is classified as having a high risk tumor.

7. The method of claim 3, wherein said candidate compound is selected from the group consisting of a small organic molecule, an intra-antibody, a peptide and a polypeptide.


SEQUENCE LISTING (1 sequence)

SEQ ID NO: 1 - protein, 209 aa, Homo sapiens
MSNLKPDGEHGGSTGTGSGAGSGGALEEEVRTLFVSGLPVDIKPRELYLLFRPFKGYEGSLIKLTARQPVGFVIFDSRAGAEAAKNALNGIRFDPENPQTLRLEFAKANTKMAKSKLMATPNPSNVHPALGAHFIARDPYDLMGAALIPASPEAWAPYPLYTTELTPAISHAAFTYPTATAAAAALHAQVRWYPSSDTTQQGWKYRQFC